(19) United States
(12) Patent Application Publication
(10) Pub. No.: US 2005/0260277 A1
(43) Pub. Date: Nov. 24, 2005

(54) Method and formula for anti-tumor and anti-matastatic effect

(76) Inventor: Brian C. Giles, Scottsdale, AZ (US)

Correspondence Address: JOHNSON & STAINBROOK, LLP, 3558 ROUND BARN BLVD., SUITE 203, SANTA ROSA, CA, 95403, US

(21) Appl. No.: 10/469,568

(22) PCT Filed: Feb. 28, 2001
(86) PCT No.: PCT/US01/06672

§ 371 Date: Aug. 28, 2003

(51) Int. Cl. A61K 38/23; A61K 31/4965; A61K 31/59; A61K 31/21; A61K 33/04; A61K 33/14; A61K 33/42

(52) U.S. Cl. 424/601; 424/702; 424/677; 514/2; 514/167; 514/255.06; 514/514

(57) ABSTRACT

The invention discloses an expeditious method and formula for treating cancer patients that reaulst in tumor suppression and remission, the suppression of mobilization and adhesion of cancer cells, and repression of pathogens, such as infectious bacteria and viruses. Administration is by oral ingestion or direct injection into tumors or intravenous drip etc. The invention employs control of intracellular and extracelular ionic physiology by administering alkaline salts, thereby restoring localized and/or systemic cellular ionic physiology, depriving cancerous cells of their ability to grow rapidly, and simultaneously normalizing their local environment, thereby inhibiting angiogenesis, enabling immune responses, and simultaneously reducing pain. The method further promotes correction of acidotic conditions often associated with cancer and other diseases, thereby potentiating biological functions of immunity and repair.

### TECHNICAL FIELD

The present invention generally relates to the field of pharmacology and drugs and more specifically relates to the in vivo method and formula for administration of a therapeutically effective dosage to cancer patients of therapeutic agents for anti-metastasis activity and malignant and non-malignant tumor remission and suppression and anti-adhesion and anti-mobilization by altering systemic, localized and/or cellular ionic physiology. For prevention of cancer relapse and tumor re-growth while simultaneously providing substantial pain relief.

### BACKGROUND ART

Cancer cells are different from normal healthy cells in several respects. One way in which virtually all cancer cells differ from normal healthy cells is that cancer cells derive a large proportion of their energy from glycolysis. Normal healthy cells utilize oxidative metabolism in which only a small proportion of energy is derived from glycolysis. Only in exceptional cases, for example during bursts of extreme muscular effort, will normal healthy cells derive a large proportion of their energy from glycolysis, but in the normal state, they use oxidative metabolism. Thus energy metabolism provides a general distinction between normal cells and cancer cells.

One characteristic of the aberrant energy metabolism of cancer cells is that they produce acids such as lactic acid, etc. This results in a pH in the immediate vicinity of the cancer cells (pHe, or pH on the exterior of the cell, as compared to systemic pH, which is the overall pH of the biological system) that is substantially lower than normal pH (The negative logarithm of the Hydrogen ion activity: pH=−log(H+)). Cancers exhibiting lower pHe values are generally rapidly growing and more likely to be fatal to the patient. Low pHe can serve as a trigger for vascularization, thereby enhancing blood flow to the tumor mass. Low pHe decreases the efficacy of the immune response to cancer. The aberrant energy metabolism typically has a lower energy charge (ATP/(ADP+Pi)) than normal. Cancer cells usually have an intracellular pH (pHi, or pH in the interior of the cell) that is lower than normal. That is, they contain more hydrogen ions than normal cells. Further, cancer cells typically have cellular distributions of ions that are different from normal cells typically showing excess internal sodium and grossly excess internal calcium, often with a deficiency in internal potassium. Cancer cells typically have ion fluxes that are different from normal cells. Cancer cells typically have membrane electrical potentials (inside relative to outside) less electro-negative than normal cells. Aberrant ion concentrations such as low pHi, high internal sodium or high internal calcium can induce apoptosis and/or can result in recognition of the cancer cell by the immune system.

Development of cancer involves a balance between the growth of neoplastic cells and their destruction by regulatory processes. The genetic changes accompanying carcinogenesis have attracted great interest, and much is known about them. Such changes are prerequisite to the development of disease, but are not sufficient to overcome the body's natural defenses. Thus cancer can be prevented by treatments which potentiate the body's natural defenses such as immune response and apoptosis, so the balance between cancer development and cancer elimination is shifted to promote cancer elimination.

The key to using aberrant energy metabolism as a way to specifically target cancer cells is to find a formulation and therapeutic treatment method that has little or no toxicity to healthy normal cells, but renders cancer cells nonviable. An ideal therapeutic treatment method will also reduce the acidification produced by the cancer cell mass, so that physiologic pHe approaches 7.37 to 7.40. If pHe is close to normal, metabolic function is not compromised by acidosis, anti-cancer activities of the immune system function in an optimal biochemical/ionic environment, and the promotion of new blood vessels, which occurs in response to reduced pH will not occur. Sufficient alkalization will also mitigate the acidotic effects of tumor necrosis. Such a treatment will clearly be beneficial in a wide variety of acidotic related degenerative diseases.

Cesium and rubidium are alkali metals with chemical and physical characteristics similar to potassium, but with greater atomic weights. Their availability in the biosphere, i.e. food and water, is virtually non-existent. Potassium is the major internal cation of living cells, and potassium ion currents are central to the ionic physiology of cells. Mammalian cells generally respond to conditions that induce glycolytic energy metabolism with large potassium fluxes. Transmembrane fluxes and cellular accumulation of cesium and rubidium are governed by the same cellular mechanisms as those which govern potassium movements, but they move at slower rates and accumulate to different degrees, thereby altering the ionic physiology of the cell, including inhibition of transmembrane movement of potassium.

Prior Art Treatment Modalities

1. Surgery: Prior art treatment of cancer relies heavily on surgical removal of the tumor load. There are stresses associated with surgery, as well as high costs and a high risk of metastasis, and it is extremely difficult to be certain that cancerous tissue is completely removed (often residual cancer cells survive and mobilize), and usually additional therapies are required. Surgery further requires separate drugs for pain reduction that promote chemical and psychological addiction.

2. Chemotherapy: The prior art has primarily consisted of treating serious degenerative diseases such as cancer with pharmo-kinetic osmotic pharmaceuticals or radiation, and does not take into consideration the biological system's electro-physiological responses, and the treatments additionally further contribute to acidotic state, inhibit or destroy cells that normally undergo rapid cell division and/or otherwise compromise the physiological well-being of the patient.

These prior art methods and drugs are inherently slow and ineffective, particularly in terminal or late stage cancer, and have serious side effects, such as antigenic effect, and provide a very narrow therapeutic window, and adversely affect Ionic Physiology and interfere with the essential pH balance and critical enzymatic activity and assimilation of essential nutrients etc., thus substantially reducing the metabolic function and potential life span of the cells and organs, further reducing their biological function and effectiveness.

3. Radiation: The prior art use of radiation causes permanent damage and further contributes to the accumulation of acidic toxins, compromising the biological environment.

4. Therapies related to Ionic Physiology Therapy: A few chemicals have been suggested as anti-tumor agents based on the unique characteristics of cancer energy metabolism. For example, inhibitors of Na/H exchange and of the membrane proton pump have been suggested as anti-tumor agents, however these show only modest anti-tumor effects. They are distinct from and inferior to the subject of the present invention.

Some preliminary work has been done using cesium and rubidium based therapies. This work was undertaken without benefit of recent advances in understanding of the ionic consequences of the unique energy metabolism of cancer cells. Despite promising early results, only with previously unavailable technology is it possible to correctly formulate and accurately therapeutically administer reliable dose and therapeutic treatments using cesium and/or rubidium.

5. Disease caused by microorganisms: Viruses, bacteria and other infectious microorganisms can build up resistance or even immunity upon exposure to multiple antibiotics, which thus eventually become ineffective. Microorganisms often have an acidotic energy metabolism that can be targeted by ionic physiology, eliminating the possibility of the development of resistance to the drug activity.

The biological environment of all cancerous cells has a very narrow and specific viability zone limited to a narrow pH range and oxidation-reduction potential (ORP) (FIG. 1). Note, however, healthy human cells optimally function in a pH range and ORP outside the cancer's biological environment. By promoting removal of acids from the body fluids, the method and formula will move those fluids toward an ORP and pH in a range consistent with aerobic, homeostatic metabolic functions as they circulate through the biological system. Thus, subsequently eliminating the residual cancerous cells and tumor mass and other accumulated acidic substances from the system through normal metabolic discharge (kidney, respiration, digestive tract). Such a response is instrumental in prevention of disease relapse and tumor re-growth.

Degenerative diseases reveal the same disorders as acute maladaptive reactions which is an oxygen deficit, acid-hypoxia biological environment. The readjusted ionic physiology also reduces pain and swelling and a wide variety of disorders associated with these conditions. Thus electro-negative charge reduces the excessive excitability of neurons, processes the stressful biological inflammatory complex, such as super oxides, peroxides, etc., thus normalizes and stabilizes the pHi and processes toxins.

### OBJECTS AND ADVANTAGES OF THE INVENTION

The present invention is to disclose a non-toxic pharmaceutically acceptable drug which can be administered to humans or other mammals suffering from cancer, to increase pHe and pHi, and to diminish systemic acidity and therapeutically treat metastatic tumors systemically and at the primary tumor site or sites with extremely low toxicity. Note that such extremely low toxicity is very rare or non-existent in the “prior art” anti-metastatic and anti-carcinogenic inducing agents. The invention employs a method of manufacture and formulation that includes use of electrolyzed saline that results in a precise, pharmaceutically acceptable formulation with optimal availability of the active ingredients that can function effectively as a stand-alone treatment or a therapeutically effective adjunct in conjunction with a wide variety of prior art cancer treatments, such as surgical intervention, radiation, or chemotherapy, etc. Additionally, the cancer cells most susceptible to the therapeutic method and formulation are those which have the most acid-producing metabolisms, the most rapid proliferation rates and which are frequently the most recalcitrant to conventional prior art treatments such as chemotherapy and/or radiation and surgery, so cancers which have survived other therapies are likely to be susceptible to the present invention. The invention further provides promotion of hydration of body fluids and stimulation of excretion of acidic toxins. Cancer cells cannot develop resistance to the treatment/formulation as they have to many prior art therapies. By reducing or eliminating the acidification in the vicinity of the tumor cells, the growth of new blood vessels into the tumor is repressed, since formation of new blood vessels occurs in response to low pH. Dosages are adjusted to fall within targeted pHi and pHe ranges, providing a controllable degree of efficacy, so that malignant and non-malignant tumor stabilization and remission and elimination occurs in a predictable and gradual manner, avoiding the distress or mortality that can accompany tumor necrosis. Alteration of ionic physiology reduces the secretory activity of the cancer cells. This both reduces their ability to secrete or reject anti-cancer drugs, the basis for multiple drug resistance, and it also inhibits the ability to secrete tumor proteases which promotes metastasis.

A method is specified whereby efficacy of treatment can be assessed in a particular patient, by observations relating to cancer ionic physiology such as acid production, lactate production, calcium accumulation, sodium accumulation etc., that allow predictable adjustment of dosage and assessment of efficacy of the therapy. Additionally, it is well suited for use as the first selection for intervention, providing substantial pain reduction or elimination and cancer remission, reserving costly testing and other therapies only for recalcitrant cancers, effectively stopping the localized and systemic acidosis cycle, providing a fast-acting highly effective cost effective formulation for therapeutically treating a wide variety of cancers and providing a reduction of the effective dose of active ingredients.

And the prevention and/or elimination of cancer growing environments, compatibility and/or synergistic with a wide variety of prior art therapies, providing maintenance of beneficial inter-cellular changes in the ionic environment.

### DISCLOSURE OF INVENTION

The present invention provides anti-metastatic effect and remission from cancer and inhibits the ability to secrete tumor protease that promotes metastasis activity and is highly effective in the prevention of cancer viability environment. It can be used alone or in combination with surgical, radiation or chemotherapies, etc., as a short-term therapeutic treatment or long term maintenance, employing a fundamentally unique method of treating and a previously unavailable formulation and effective method for its manufacture and use is disclosed, employing a non-toxic formula to intercept the initiating source of the cancer formation process on a cellular level. The method and formula is more physiologic, preventing rebound acidosis, acidic gastritis and heart failure, converting cancerous cells and tumor mass to nutriants available for metabolism by healthy cells, obtaining therapeutic efficacy in treatment of one or more malignant and/or non-malignant (benign) tumors myoma, adenoma, polyps, cysts, and systemic (metastasis) cancers simultaneously. The cost effective aqueous based electrolytically processed formula promotes disease resistance in the intracellular environment, obtaining ionic changes in the intracellular environment, changing the chemistry of the cell, including pHe and pHi, and safely simultaneously killing a wide variety of cancer cells and alkalinizing the ionic environment, altering systemic, local and/or cellular physiology. The action of cesium and/or rubidium includes effects secondary to the inhibition of the large transmembrane potassium movements resulting from hypoxic energy metabolism. Such effects include alterations in pH control, excessive sodium accumulation, diminished membrane electrical potential and diminished capacity of sodium/calcium exchange mechanisms. Cells in conditions of normoxia have small potassium currents that can be maintained even in the presence of cesium or rubidium. The treatment and formulation has cancer-killing activity (tumor stabilization, suppression and remission) under conditions that are virtually non-toxic for all healthy cells in the biological system. Residual malignant cells may lead to disease relapse. The present invention is a particularly useful method of eradicating residual tumor cells, such as following surgical intervention, chemotherapy and/or radiotherapy etc., by obtaining beneficial changes in the cellular environment. Advantages include speed of efficacy, generally ranging from 15 to 30 consecutive days, also providing a high degree of efficacy against secondary infections, often a major cause of death in cancer patients, and improved hydration and oxygen availability combined with elimination or substantial reduction of pain.

The invention has a very high efficacy to toxicity ratio, thus making it possible to conduct a portion of the cancer treatment on an outpatient basis, resulting in substantial cost savings.

The active ingredients can be formulated for intravenous administration suitable for comatose or late stage terminal cancer patients, or formulated for the injecting of effective amounts of the balance solution suitable for earlier stage cancer patients, and in some cases suitable for outpatient formulation as an oral self-administration, or formulated as a therapeutic maintenance dosage for patients at risk of cancer relapse (genetic, dietary, environmental, etc.) or at high risk of developing cancer, or formulated in a maintenance dose for the prevention of the development of degenerative acidotic conditions.

1. Formulations for Ionic Physiology Therapies

ALKALI METAL SALTS. The principal active ingredients consist of salts of the alkali metals cesium and rubidium. The anionic moieties of the salts can be any non-toxic element or compound that does not substantially prevent biological availability of the cesium and rubidium. The cesium and rubidium compounds of the present invention may be employed either alone or in a variety of combinations to obtain the desired anti-carcinogenic and anti-metastatic activity and a variety of therapeutic effects.

ANCILLARY COMPOUNDS. Other active ingredients are chosen to complement or potentiate the effect of the alkali metals. These other ingredients may also alter the ionic metabolism of the cancer cells to make them nonviable, they may reduce the viability of the cancer cells in an unrelated way, or they may increase the tolerance of the patient to the stresses associated with cancer therapy. As an example of an ancillary compound, an inhibitor of angiogenesis may be used to complement the use of the alkali metals. Different ancillary ingredients may be chosen for the treatment of other diseases, and for the prevention of other diseases.

WATER. Water used in the manufacture of the formulation may be treated to render it pure, sterile and/or to enhance the availability of the active ingredients. In particular, water may be treated, with adequate duration and voltage, electrolytically to modify chemical and physical parameters for manufacture.

Water treated electrolytically sufficiently processed to have a negative redox potential, enabling it to neutralize electrophilic toxins, and maintaining a redox environment in which the active ingredients are available and highly effective. The water is preferably processed to have a surface tension in the range of 55 to 68 dynes per cm², most preferably 60 to 68 dynes per cm², and ORP in the range −350 to −560 millivolts, and preferably a pH in the range of 8.5 to 9.7.

2. Use in Treatment of Cancer

EFFICACY. Efficacy of the formula and therapy is monitored by standard medical observations and by observations particular to ionic physiological therapy. Such observations would include the monitoring of pHe, pHi and systemic pH.

TOXICITY. Observations and therapies related to tumor cell necrosis rates and systemic acidosis are included to prevent toxicity depending on the method of delivery.

PHYSIOLOGICAL STRESS. Observations and therapies related to physiological stresses of Ionic Physiology therapy are included to prevent therapy-related stress.

3. Use in Treatment of Disease

Observations related to efficacy may be specific to a particular disease, but they are known to those skilled in the physician's art for any known disease.

4. Use in Prevention of Cancer

The efficacy of preventative treatments can be assessed by observations of a statistical nature, well known to those skilled in the art. The therapy is theorized to prevent the development of cancer by reducing the ability of pre-cancerous cells either to acidify their surroundings or to use glycolytic energy metabolism for rapid replication, or both. The efficacy of the treatment in an individual patient can thus be assessed by observing the response to a challenge with an agent or agents which induces acid production on a cellular level, for example by induction of a transient chemical hypoxia. Alternatively, other artifacts of ionic metabolism can be measured at a cellular level, such as cytoplasmic pH. Necrosis related toxicity is not a factor in preventative application or use. Doses used for preventative purposes are lower and hence there are no significant stresses associated with ionic physiology cancer prevention.

### BRIEF DESCRIPTION OF THE DRAWING

FIG. 1 depicts the ORP/pHe (intercellular fluid) and the carcinogenic environment of a wide variety of cancers.

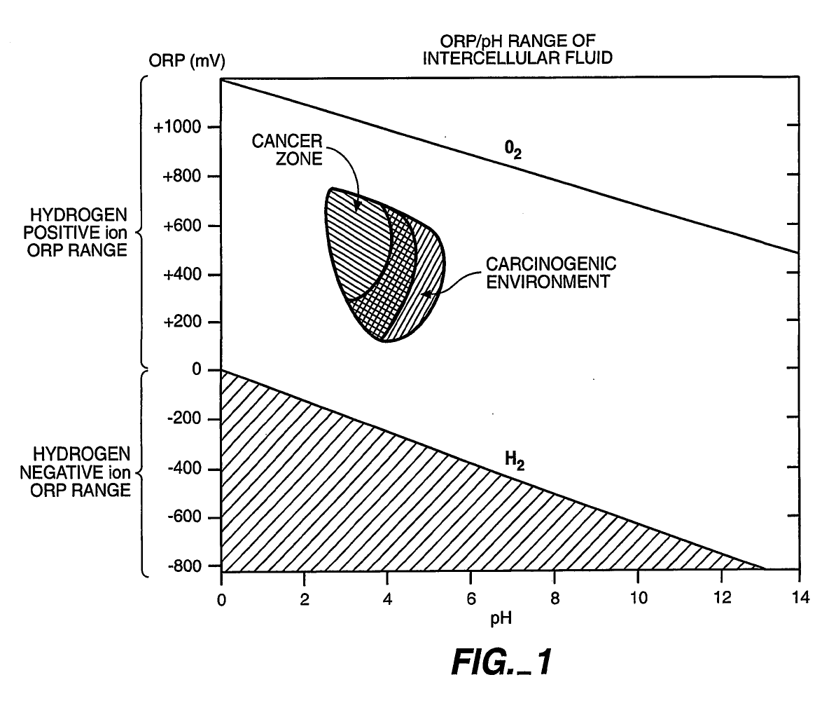

### BEST MODE FOR CARRYING OUT THE INVENTION

The preferred embodiment provides an in vivo method and formula for ionic therapeutic treatment of serious degenerative diseases such as cancer, for effective tumor stabilization, suppression and remission, as an example, but not limited to: lung cancer, breast cancer, colon cancer, prostate cancer, liver cancer etc.

This invention discloses a previously unavailable method and formula of using alkaline salts in an electrolyzed solution which meet certain electrochemical and electro-physiological requirements, for treating human patients, or other mammals suffering from cancer, with one or more malignant or non-malignant (benign) tumors, providing to the cells an effective or therapeutically sufficient dose of cesium and/or rubidium ions. It further discloses a method and formulation for a preventative and maintenance dose for treatment for a wide variety of cancers.

The method and formula alkalinizes the systemic and localized acidity levels of the extracellular fluids in cancerous tumors and changes the intracellular ionic environment of the cells. Cesium and rubidium ions taken up into the cell tend to be released very slowly. Some of these ions remain in the cell for the life-span of the cell, so they have longer persistent effects. When the acidity of a cancerous tumor is reduced to a more physiologically normal level, the patient's metabolic function and immune system (including antibodies, macrophage cells, etc.) function more effectively and reduce and inhibit cancer cells' replication in the tumor mass. Such a response is instrumental in prevention of disease relapse.

A high percentage of cancer deaths are due to a variety of secondary infections, usually by pathogens such as bacteria and other infectious microorganisms. If a patient's immune system is suppressed by an acidotic biological environment, either bacterial-induced or tumor-generated, then the treatment described herein will stimulate the immune system by restoring the pH and pHe to a homeostatic level, thereby helping the patient to resist a wide variety of secondary infections as well as promoting the immune function response.

Method of Manufacture

PRINCIPAL ACTIVE INGREDIENTS. This invention utilizes salts of cesium, rubidium, or in combination, in its manufacture. Both cesium and rubidium salts can be employed, for example, but not limited to, cesium chloride and rubidium chloride. This invention discloses an in vivo method and formula of treating cancer, employing an alkaline salt solution formed by the following formula: MA, where MA substantially dissociates in water solution to form M+ and A−. M is the alkali metal moiety, which may be cesium and/or rubidium. A is the anionic moiety, which may be any compatible non-toxic inorganic species such as chloride, sulfate, carbonate or phosphate; or it may be any non-toxic organic species such as lactate, citrate or acetate.

In the event that it is desired to combine the alkali metal moiety with an anionic moiety with which it is not readily available, this can readily be accomplished. For example, the hydroxide of the alkali metal can be combined with the acid form of the desired anion, thus:
MOH+HA forms MA+H₂O

In the case of acids that can dissociate more than one hydrogen ion, the final product may be partially protonated, for example, MHCO₂ or M₂CO₂, either the bicarbonate or carbonate salt of carbon dioxide. The final product can be controlled by controlling the stoichiometry of the reaction, or by any known manufacturing process to obtain a desired final pH.

When it is desired to decrease systemic acidity, carbonate or an organic species that can be metabolized are preferred. For example, citric acid can be used to neutralize a solution of cesium hydroxide until a pH near neutrality is obtained, or precise amounts of cesium hydroxide can be mixed with predetermined amounts of citric acid so that on dissolution a predetermined physiologic pH will be obtained. If tumor metabolism is monitored by lactate or lactate dehydrogenase (LDH) measurements, it may be preferable to avoid use of lactate to minimize background lactate or LDH signals. For oral formulation and administration, palatability will influence choice of anion(s), and the flavoring agent or agents employed.

The proportion or ratios of cesium to rubidium employed will be governed by considerations of efficacy in tumor stabilization, suppression and remission and physiological stress in the patient. In the event of physiological stress from high doses of one ion, a variety of combinations can reduce stress effects while retaining sufficient therapeutic effect.

If necessary the active ingredients may be formulated to be released in the optimal part of the digestive tract to avoid nausea while retaining availability. Or may be designed to provide delayed or controlled release using formulations and techniques which are know in the art.

SECONDARY ACTIVE INGREDIENTS. These ingredients are chosen to complement or potentiate the action of the active ingredients. Some examples of potentiating ingredients are given to instruct the physician in the principals of their selection and are not intended to exclude other ingredients not mentioned. Potentiation of cesium/rubidium action can be accomplished by inclusion of ingredients that enhance the tendency towards apoptosis induced by ionic physiology. Examples are compounds that stimulate calcium accumulation, such as calcium supplements and magnesium, vitamin D, selenium salts, calcitonin, calcium ionophores, etc., compounds that defeat the elimination of sodium from cancer cells such as monensin or inhibitors of sodium/potassium exchange, compounds that alter pH regulating characteristics such as nigericin, amiloride and its derivatives, 4,4-diisothiocyanostilbene 2,2-disulfonic acid and bafilomycin. Further examples are compounds that decrease glucose utilization by tumor cells, such as lonidamine, and compounds that independently increase the activation of apoptosis. Another class of ingredients which potentiate the activity of the primary active ingredients are those which stimulate or support the immune system, especially those which may be deficient as a secondary consequence of cancer, such as magnesium, zinc, vitamin B2 and B12. Ingredients that complement the cesium and/or rubidium therapy are those that act by unrelated means but which may be useful in reducing cancer viability. These include the wide variety of chemotherapies that do not target ionic physiology. Because cancer development is a balance between reproduction and death of cancer cells, any additional ingredient that reduces reproduction or enhances cancer cell death can potentially be useful in the case of cancers recalcitrant to treatment with cesium and/or rubidium therapy alone. Use of toxic chemotherapies is minimized, and preferably done under careful medical supervision. An additional class of ingredients which complement the action of rubidium and/or cesium therapy are those which minimize the toxic effects of tumor necrosis. These include hydration, other alkalizing treatments, treatments that reduce the toxicity of tumor necrosis and nutritional and dietary intervention or supplementation appropriate to the physiological stress associated with cancer or tumor necrosis. An additional class of ingredients which complement the action of rubidium and/or cesium therapy are potassium and other mineral supplements and dietary supplements and anti-oxidants which compensate for potassium and other losses which may occur due to the mild diuretic effect of the therapy. Mineral supplements including trace minerals and ions are also used to obtain and maintain the desired pH range of bodily fluids and cellular metabolism.

WATER. If a solidified crystalline formation of the salt or salts described herein is desired for purposes such as shipping or storage or oral administration, it can be prepared by conventional methods, containing buffered salt or salts.

If preferred, the active ingredients may be orally administered without previous dissolution, or they may be prepared as a solution suitable for ingestion or injection, using an aqueous carrier liquid. For example, solutions for injection should be prepared with a chemical composition that renders them balanced and acceptable for injection. Typically, injectable solutions will be comprised of active ingredients in a sterile buffered saline solution isotonic to blood.

Water used may be from any source of suitable purity. As an example, but not limited to, the preferred method of manufacture is to use water processed by means such as electrolytic treatment, whether 100% throughput or separate anodic and cathodic output streams, employing adequate voltage/current for a period of time (exposure) or external energy fields (electromagnetic, magnetic, radiation, sonic, etc.) which gives the advantage of pharmaceutically acceptable dose uniformity and is suitable for manufacture with a wide variety of concentrations useful for a wide variety of applications. The electrical energy fields alter the electro-viscous characteristics of the water molecule cluster, restructure the water and thus alter the characteristics of the water. A combination of adjustments such as the actives content, water flow-rates in the reaction chambers, fluid pressure differences, and controllable voltage/current intensity and exposure time are made to obtain an accurate pharmaceutical manufacturing process. Controlled concentrations of dissolved suspensions of the active substances are added, creating an aqueous mixture that is electrolytically processed, by adding the alkaline salts to the restructured aqueous mixture which is and is of an effective quantity or concentration in treating a wide variety of cancers which are susceptible to such treatment.

The high-intensity field electrochemically restructures the aqueous carrier liquid activated into smaller metastable hydrogen bonds between the water clusters forming or assembling around the mineral ions reducing their size by approximately 50%, or process to obtain smaller clusters, if necessary lowering the viscosity and surface tension of water. As an example, surface tension reduction is from 73 dynes per cm² to between 55 and 68 dynes per cm². Note, lowering surface tension improves solubilization and ionic availability. Lowering the viscosity of water improves systemic hydration etc.

The active water soluble salts (cesium and/or rubidium) in the formula are introduced via high speed computer controlled, injectors controlling the precise concentrated injection dosage and water flow rates in the formulation, by injecting into the fluid stream just prior to the electrolysis reaction chamber/chambers where the water is electrochemically restructured. Note, some mineral salts and activators contained in the concentrated formula are injected after the electrolysis process. Water production parameters are chosen so that the product has a physiologically acceptable value of pH and ORP to be virtually non-toxic, to provide optimal availability of actives in the solution and to support hydration and to counteract the acidosis cycle. The formulation ORP values are −350 to −700 millivolts most preferably −350 to −560 millivolts to avoid possible damage to healthy cells and tissues. For oral ingestion, the pH may range from 8.5 to 9.7, preferably 8.6 to 9.5. The formula and active ingredients are generally expected to be approximately 2× or more as available as with non-electrolyzed water preparation, depending on the water characteristics and the physiological condition of the patient.

Method of Use

MODES OF ADMINISTRATION. The alkaline salt solutions of this invention can be administered directly to the bloodstream, by any suitable means such as (but not limited to) periodic injections, intravenous infusion, rectal, neogastric, transdermal, peritonial, subcutaneous, intramuscular, intrathecial, sublingual, or topical administration, use of an implanted osmotic mini-pump or other slow-release methods or devices, etc. As used herein, the term “injection” includes any such method of introducing the actives or compounds directly into the blood circulation. The injectable formulations preferably should be isotonic with blood, and should have a pH value of approximately 7.3 to 7.4. Such formulation may be designed to provide delayed or controlled release using formulations and techniques which are known in the physician's art. Injection may be to other sites, such as direct injection into or near a tumor or tumors, etc.

The alkaline salts described herein can also be administered orally if desired, or alternatively in a tablet or capsule, or as a nutrient additive. Oral administration is most preferably simultaneously ingested with a suitable carbohydrate.

Other formulations may be used if required such as pharmaceutically acceptable compositions containing the active ingredients which may take the form of gels, oils, bandages/dressings, topical lotions, douche solutions, suppositories, colon irrigation solutions, sublingual drops, or drop dispersions.

In general, the prescribed dosage required for therapeutic efficacy will be dependent on such factors as the patient's weight, age, diet, gender, physical symptoms and condition, duration and frequency of administration, chosen route of administration, and the variety of cancer or cancers and its stage of advancement. The lethal limit (cesium) is about 10 mEq/kilogram in mice, with no distress but some depression of activity at 5 mEq/kilogram. The lethal limit (rubidium) is about 1.93 mEq/kilogram in mice.

As a general guide, the effective therapeutic dosage range expressed as amount of cesium is 0.1 to 5 mEq per kilogram daily for cancer therapy, 0.005 to 0.1 nmEq per kilogram per 24 hrs. for cancer prevention, and 0.00001 to 0.01 mEq per kilogram per 24 hrs. for prevention of cancer related degenerative diseases. Doses over 1 mEq per kilogram should be used only if absolutely necessary, and with careful monitoring for stress symptoms. Juvenile doses should be lower, generally about ½ of the adult range, depending on weight, etc. Cesium or rubidium should not presently be administered to pregnant or lactating women or infants without further studies. Direct tumor injection doses can be considerably smaller, utilizing up to 300 mg per kilogram of tumor mass daily. The degree of anti-tumor action required, the degree of alkalization required, and the presence of any stress effects thus determines the dosage or amounts of cesium and/or rubidium used in therapy. The optimal effective formula and dose is adjusted as therapy progresses. As an example, but not limited to, a patient suffering from acute acidosis may be treated principally for that condition with rubidium, followed by a gradual increase in cesium or any therapeutically effective combination, to obtain anti-tumor activity sufficient to give a non-stressful degree of tumor necrosis. Preferably, the dose is selected to give only mild efficacy to begin with and increased as appropriate, to avoid shock due to excessive release of acid toxins, for example due to large tumor volume necrosis. Note, the therapy is most effective if diet is nutritionally adequate and does not contribute to acidotic stress. As an example, dietary foods and beverages with pH below 2.5 should be completely eliminated, and foods with pH below 3 and beverages and foods whose low pH results from mineral acids such as phosphoric acid should be minimized or substantially reduced. Note, a neutral or slightly alkaline diet is encouraged during therapy.

EFFICACY. Regardless of the mode of administration, the patient's tumor or tumors should be monitored, as well as vital signs such as temperature and blood pressure. The patient's saliva, urine and blood pH should be monitored during the treatment process, and the dosage should be appropriately adjusted to accommodate the needs of the patient's physiological condition to systemically and at the tumor site partially or wholly restore the proper pHe to near-physiological levels of approximately 7.3 to 7.4, resulting in release of stored acids inside and outside the cells. Whenever possible, the pH of the fluid inside the tumor, or tumors, or the pH of blood emerging from a tumor, should be monitored in the most accurate manner practicable. As an example, magnetic resonance spectroscopy or other suitable methods including tissue sampling and analysis can be used to monitor pHe, pHi, other indicating features of ionic physiology such as tumor sodium, potassium, magnesium and calcium levels, and metabolites such as lactate. Alternatively, near infrared spectroscopy can be used to non-invasively monitor pH. Further indications of efficacy are tumor stabilization, shrinkage or remission, and presence in blood or other body fluids of markers of tumor necrosis. A dosage and formulation that result in reduction in pHe and in a normal, as opposed to cancerous, ionic response to glycolytic metabolism are a short-term indication of effectiveness of the therapy. Note that the method and formula is suitable for large volume tumors that must be confirmed by tumor regression. A lack of response by pHe and other indicators indicates an insufficient dosage.

STRESS. Excessive doses of rubidium and cesium salts can cause physiological stress, for example from the mild diuretic effects, or as a result of potassium depletion, or from excessively low blood pressure, or from excessive systemic alkalization. In cases where maximal efficacy is required during therapeutic treatment, the upper limit to dosage is set by stress symptoms. Note, the maximum dosage must be below the point at which perturbation of electrolyte balance causes damage. The pH measures noted above for efficacy will provide information useful for the practicing physician in assessment of physiological stress. Blood pH should not rise above 7.4, saliva pH should only briefly rise above 7.7 and urine pH should not rise substantially above 7.1 or below 5.0 or, briefly, below 4.5. An additional symptom of excessive pH rise is sore muscles and/or numbness around the mouth. This indicates a reduced dose or brief suspension of treatment to avoid excessive alkalosis induced stress and an increased dosage of potassium until the symptoms are reduced to an acceptable level. Blood potassium should not fall below tolerable levels. Creatine levels should be monitored. Dehydration should be monitored and corrected if it occurs. The patient is preferably well hydrated before initiation of treatment. Blood pressure should be monitored. Sensations of numbness indicate incipient effects on nerve tissue ionic status. Doses should not exceed those that cause very slight sensations of numbness.

As used herein, the terms “therapeutic” and “therapy” refer to a treatment which helps a patient's body fight or resist cancer, regardless of the specific mode of action of the alkaline salt or salts disclosed herein, and regardless of whether a cancer in a specific patient goes completely into remission. For example, a treatment which prolongs survival or helps ameliorate pain is highly useful, even if it cannot provide life long remission in a specific patient.

The following examples are offered to illustrate possible uses of the technology. Those skilled in medical practice will recognize that there are many variations on these formulations and methods, which will depend on the individual patient's requirements and other circumstances.

### EXAMPLE 1

Cesium and/or rubidium cancer therapy dosage for oral administration. Amounts per 4 ounces bottle of formulation in electrolyzed water solution containing cesium and/or rubidium salts and other ingredients: Cesium citrate and/or rubidium citrate, or any combination thereof, ranging from 500 mg per 24 hours to 5,000 mg per 24 hours, preferably 2,500 mg per 24 hours;
potassium (preferably as phosphate, gluconate and acetate)500-2000 mg; calcium 2,500 mg; magnesium citrate 200-1500 mg; sodium chloride; iodine; selenium (Selenomethionine) 50-200 mcg; vanadium (vanadyl sulfate) 2-10 mg; zinc gluconate 30-200 mg;
Vitamin D 2,000 to 4,000 IU; Vitamin A 2,000 to 5,000 IU; Vitamin C-(L-ascorbic acid) buffered 1,000 to 5,000 mg; malic acid 100-500 mg; CO Enzyme Q 10 ubiquinone 25-50 mg; DHEA (dehydroepiandrosterone) 5-50 mg; B3 methyl nicotinate 20-30 mg; B6 25-100 mg; and B12 20-50 mg.

Administered as 4 ounces 2 times per 24 hours. The patient should be monitored for stress and efficacy as described above, and therapy adjusted to obtain tumor remission and suppression response with minimal physiological stress. Failure to respond, either initially or after a period of favorable response, indicates that complementary or potentiating ingredients should be considered.

### EXAMPLE 2

Cesium/rubidium therapy with slow I.V. administration.

I.V. solution and dosage: The preferred embodiment generally ranges from 200 mg to 10 grams per liter CsCl, more preferably 1,200 mg per liter, and/or from 200 mg to 10 grams per liter RbCl in buffered saline made isotonic to blood. Any combination of CsCl and RbCl at the foregoing concentrations may also be utilized.

As an example, administered by continuous intravenous drip (2×) per 24 hrs. generally ranging from 250 to 1000 cc as necessary. Note, if the patient's physiological condition is life threatening such as comatose terminal stage cancer, a higher dosage may be required, as an example, 1,000 cc(2×) per 24 hrs. Patient to be kept hydrated and given an adequate diet including vitamin and mineral supplement, and an oral dietary supplement obtaining the following: potassium (preferably as phosphate gluconate and acetate) 150 to 1,200 mg; calcium 1,500-2,500 mg; magnesium citrate 200-1500 mg; iodine; selenium (Selenomethionine) 50-200 mcg; vanadium (vanadyl sulfate) 2-10 mg; zinc gluconate 50-200 mg; Vitamin D 2,000 to 4,000 IU; Vitamin A 2,000 to 5,000 IU; Vitamin C-(L-ascorbic acid) buffered 1,000 to 5,000 mg; malic acid 3-5 mg; CO Enzyme Q 10 ubiquinone 25-50 mg; B3 methyl nicotinate 5-20 mg daily; B6 25-100 mg; B 12 20-50 mg.

The patient should be monitored for stress and efficacy as described above, and therapy adjusted to give a positive response with minimal stress. Failure to respond either initially or after a period of favorable response indicates that complementary or potentiating ingredients should be considered.

### EXAMPLE 3

Cesium/rubidium tumor remission and suppression and/or long term maintenance dose.

Amounts per tablet or capsule administered once or twice daily, preferably with a carbohydrate, ranging from 250 to 1000 milligrams, preferably 500 mg daily, as an example but not limited to: cesium citrate 400 mg; rubidium citrate 100 mg; potassium (preferably as phosphate gluconate and acetate) 150 to 1200 mg; calcium 2,500 mg; magnesium citrate 200-1,500 mg; iodine; selenium (Selenomethionine) 50-200 mcg; vanadium (vanadyl sulfate) 2-10 mg; zinc gluconate 50-200 mg; Vitamin D 2,000 to 4,000 IU; Vitarnin A 2,000 to 5,000 IU; Vitamin C-(L-ascorbic acid) buffered 1,000 to 5,000 mg; malic acid 3-5 mg; CO Enzyme Q 10 ubiquinone 25-50 mg; B3 methyl nicotinate 5-20 mg daily; B6 25-100 mg; B12 20-50 mg., DBEA (dehydroepiandrosterone) 5-50 mg.

Administered as one or more tablets, depending on dose or capsule per 24 hrs. This formulation is intended for use by patients who are at high risk of reoccurrence and should be checked periodically by a practicing physician employing a medically appropriate method. Saliva pH should range from 7.2 to 7.4, preferably 7.37, to maximize genetic integrity and repair. Stress monitoring may be indicated if there is some medical condition that may be exacerbated by the therapy such as conditions relating to compromised or abnormal mineral absorption or low blood pressure.

The use of novel method and formula of the present invention have qualities that will be appreciated as this application encompasses broader and other aspects than recited in these examples.

The facts and theories discussed in this disclosure are intended to teach the reader how to use the invention. However, the theory underlying the invention is not part of the claims and the inventor does not wish to be bound by any particular theory explaining the invention. In fact, it is fully anticipated that the theory underlying the present invention will evolve as the oncological sciences develop and mature.

While this invention has been described in connection with preferred embodiments, it is obvious that various modifications, changes or substitutions therein may be made by those skilled in the art to which it pertains, without departing from the spirit and scope of the invention. Accordingly, the scope of the present invention is to be limited only by the appended claims and their legal equivalents.

1. A composition of matter comprising an aqueous alkali metal salt solution for use in the treatment of mammalian cancer, having the general formula:
MA_{(aq)}, wherein
MA dissociates in water to form M+ and A−;
M is an alkali metal selected from the group consisting of cesium and rubidium, and comprises cesium and rubidium either alone or in any combination thereof, and
A is an anion selected from the group consisting of chloride, sulfate, carbonate, phosphate, lactate, citrate, and acetate.

2. The composition of matter of claim 1 further including:
at least one substance to stimulate calcium accumulation, said substance selected from the group consisting of Vitamin D, selenium salts, calcitonin, and calcium ionophores;
at least one substance to reduce the elimination of sodium from cancer cells, said substance selected from the group consisting of monensin, and sodium/potassium exchange inhibitors;
at least one pH-modifying substance selected from the group consisting of nigericin, amiloride, 4,4′-diisothioscyanostilbene 2,2-disulfonic acid, and bifilomycin, in an amount sufficient to decrease acidity at the tumor site in the patient and systemic acidity in the patient; and
at least one substance to depress glucose utilization by tumor cells.

3. The composition of matter of claim 1 further including:
at least one substance in an amount sufficient to increase the activation of apoptosis in the patient;
at least one substance in an amount sufficient to stimulate the immune system, said substance selected from the group consisting of magnesium, zinc, Vitamin B2 and Vitamin B12;
at least one substance that complements cesium and/or rubidium therapy by unrelated means but which may be useful in reducing cancer viability, including compounds well known in the art and commonly used in chemotherapies that do not target ionic physiology; and
at least one substance in an amount sufficient to compensate for potassium loss due to any diuretic effect of the therapy, selected from the group consisting of potassium, anti-oxidants, and mineral supplements including trace minerals.

4. The composition of matter of claim 1 wherein said solution is suitable for oral administration twice daily in four ounce doses, said alkali metal salt is cesium citrate and/or rubidium citrate, or any combination thereof, in an amount ranging from 250 mg to 2,500 mg; and wherein said solution further includes 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandroststerone); 10 to 15 mg B3 methyl nicotinate; 12.5 to 50 mg B6; and 10 to 25 mcg B12.

5. The composition of matter of claim 1 wherein said alkali metal salt is cesium chloride and/or rubidium chloride, either alone or in any combination thereof, in an amount ranging from 200 mg to 10 grams alkali salt per liter of water, and said solution is buffered and isotonic to blood, said solution being suitable for administration by intravenous drip twice per 24 hours in an amount ranging from 250 to 2,000 cc, depending on patient needs.

6. The composition of matter of claim 5 further including 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandrosterone); 10 to 15 mg B3 methyl nicotinate; 12.5 to 50 mg B6; and 10 to 25 mcg B12.

7. The composition of matter of claim 1 wherein said alkali metal salt comprises 400 mg cesium citrate and 100 mg rubidium citrate; and further comprises 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandrosterone); 10 to 15 mg B3 methyl nicotinate; 12.5 to 50 mg B6; 10 to 25 mcg B12, and wherein the aqueous solution is processed for formulation into dry tablet or powdered form for oral administration twice daily to a cancer patient.

8. A method of treating mammalian cancer, comprising the steps of:
administering to a patient a therapeutically effective quantity of an aqueous alkali metal salt solution, wherein the alkaline salt has the general formula:
MA_{(aq)} and MA dissociates in water to form M+ and A−;
M is an alkali metal selected from the group consisting of cesium and rubidium, and comprises cesium and rubidium either alone or in any combination thereof; and
A is an anion selected from the group consisting of chloride, sulfate, carbonate, phosphate, lactate, citrate, and acetate.

9. The method of claim 8 wherein the aqueous alkali metal salt solution further includes at least one substance to stimulate calcium accumulation, said substance selected from the group consisting of Vitamin D, selenium salts, calcitonin, and calcium ionophores, and at least one substance to reduce the elimination of sodium from cancer cels, said substance selected from the group consisting of monensin, and sodium/potassium exchange inhibitors.

10. The method of claim 8 wherein the aqueous alkali metal salt solution further includes:
at least one pH-modifying substance selected from the group consisting of nigericin, amiloride, 4,4′-diisothioscyanostilbene 2,2-disulfonic acid, and bifilomycin, in an amount sufficient to decrease acidity at the tumor site in the patient and systemic acidity in the patient;
at least one substance to depress glucose utilization by tumor cells, and further includes at least one substance in an amount sufficient to increases the activation of apoptosis in the patient;
at least one substance in an amount sufficient to stimulate the immune system, said substance selected from the group consisting of magnesium, zinc, Vitamin B2 and VitaminiB12; and at least one substance in an amount sufficient to compensate for potassium loss due to any diuretic effect of the therapy, said substance selected from the group consisting of potassium, anti-oxidants, and mineral supplements including trace minerals.

11. The method according to claim 8, wherein the alkali metal salt solution is orally administered.

12. The method according to claim 8, wherein the alkali metal salt solution is administered by injection.

13. The method according to claim 8, wherein the alkali metal salt solution is administered by introduction of said substance into a bodily cavity.

14. The method according to claim 8, wherein the alkaline salt solution is applied directly to cancerous neoplasms.

15. A method of treating mammalian cancer, comprising the steps of administering a therapeutically effective dose of an aqueous alkali metal salt solution, wherein the alkali metal salt is selected from the group consisting of cesium citrate, cesium chlroide, rubidium citrate, and rubidium chloride, either alone or in any combination thereof, in an amount ranging from 250 mg to 2,500 mg; and wherein said solution further includes 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandroststerone); 10 to 15 mg B3 methyl nicotinate; 12.5 to 50 mg B6; and 10 to 25 mcg B12.

16. The method of claim 15 wherein said alkali metal salt is cesium citrate and rubidium citrate, either alone or any combination thereof, in an amount ranging from 250 to 2,500 mg per four ounces of solution, administered orally twice daily.

17. The method of claim 15 wherein said alkali metal salt solution comprises aqueous cesium chloride and/or rubidium chloride, either alone or in any combination thereof, in an amount ranging from 200 mg to 10 grams alkali salt per liter of water, said solution buffered and isotonic to blood, said solution administered 250 to 2,000 cc per day by intravenous drip, depending on patient needs.

18. The method of claim 17 wherein the aqueous alkali metal solution further includes 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25 mg COq; 2.5 to 25 mg DHEA (dehydroepiandrosterone); 10 to 15 mg B3 methyl nicotinate; 12.5 to 50 mg B6; and 10 to 25 mcg B12.

19. The method of claim 15 wherein said alkali metal salt comprises 400 mg cesium citrate and 100 mg rubidium citrate; wherein said solution further comprises 125 to 1000 mg of a potassium salt selected from the group consisting of potassium phosphate, potassium gluconate, and potassium acetate; 1,250 mg calcium; 100 to 1,250 mg magnesium citrate; iodine; 50 mcg to 150 mcg selenomethionine; 1 to 5 mcg vanadyl sulfate; 25 to 100 mg zinc gluconate; 1,000 to 2,000 IU Vitamin D; 1,000 to 2,500 IU Vitamin A; 500 to 2,500 mg buffered Vitamin C (L-ascorbic acid); 50 to 250 mg malic acid; 12.5 to 25-mg COq; 2.5 to 25 mg DHEA (dehydroepiandrosterone); 10 to 15 mg B3 methyl nicotinate; 12.5 to 50 mg B6; 10 to 25 mcg B12; said method including the further steps of formulating said solution into dry tablet or powdered capsule form for oral administration, said tablet or capsule being suitable for the long term treatment of mammalian cancer.

20. The method for treating mammalian cancer of claim 8 further including the step of monitoring pH and adjusting the therapy so that the systemic pH, the tumor pHe and the tumor pHi fall within a predetermined range.
